(19) United States
(12) Patent Application Publication
(10) Pub. No.: US 2016/0007722 A1
(43) Pub. Date: Jan. 14, 2016

(54) System for securing items to outerwear.

(71) Applicants: Brenda Macon, Durango, CO (US); Todd Macon, Durango, CO (US)

(72) Inventors: Brenda Macon, Durango, CO (US); Todd Macon, Durango, CO (US)

(21) Appl. No.: 14/329,906

(22) Filed: Jul. 12, 2014

(51) Int. Cl. A45F 5/06; A41D 1/04; A41D 1/14; A61F 9/02; A41D 19/00; A41D 19/01; A42B 1/04; A41D 3/02; A41D 1/06

(52) U.S. Cl. CPC A45F 5/06; A41D 3/02; A41D 1/04; A41D 1/14; A41D 1/06; A41D 19/0041; A41D 19/01; A42B 1/04; A61F 9/02

(57) ABSTRACT

A system for securing gloves, mittens, hats, goggles, and other small items to the chest area of a coat, jacket, vest or shirt, or to a thigh-pocket area of pants by the use of magnets and garment clips. A magnet is disclosed and comprises a first magnet for positioning exterior on the exterior face of the chest area on a jacket, vest, coat, or shirt, or on the thigh area of pants, the second magnet being for positioning interior on the interior face of said garment, the second magnet being magnetically attracted to the first magnet. A garment clip, also known as a suspender, pacifier, or mitten clip, is disclosed and positioned and clipped on the item needing to be retained. A method includes positioning the first magnet adjacent to the exterior face of the garment, positioning the second magnet adjacent to the interior face at a location that is substantially opposite of the first magnet such that magnetic attraction pulls the first and second magnets toward each other on the opposite wall faces, and moving the garment clip now fastened to the item (such as a glove, mitten, hat, etc.) to the location of the first magnet, and bringing the item into contact with the first magnet such that the item is magnetically attracted to it, whereby the item is retained without straps, lariats, elastic, buckles, velcro, or snaps.

### BACKGROUND OF THE INVENTION

1. Field of the Invention

The present invention relates to article securing devices and more particularly pertains to a new system for securing items to outerwear that are easily mounted on and removed from the outerwear garments for permitting easy retrieval of the items.

2. Description of the Prior Art

The use of article securing devices is known in the prior art. In the context of purses or other handbags, devices have been proposed that require the mounting of one component on the purse or handbag and another component on the item to be secured, such as a key or key ring. This type of approach increases the bulkiness of the item to be secured, and can also make the item relatively difficult to remove, depending upon the technique used for securing the components together. Further, and probably most disadvantageously, some devices require a permanent or semi-permanent mounting of one of the components to the purse is required, which may damage or require alteration of the purse, but also makes it difficult if not virtually impossible to transfer the device between purses or other bags, etc.

In the context and use of magnetic gloves, there are various uses for magnets, such as to affix carpenter nails to the back of a contractor's glove; to increase the gripping strength of a glove, to put at the fingertips of neoprene scuba gloves for the purpose of gathering metal filaments under water, and for the purpose of magnetic therapy in the hand.

Finally, the use of article securing devices such as the leashes that create a connection between two mitten clips that fasten handwear to outerwear are disadvantageous to the active sportsperson who has no time or inclination to fumble with the attachment and removal of putting the clips on and off or the subsequent tangling of leashes, lariats or elastic bands.

In these respects, the system for securing items in to outerwear according to the present invention substantially departs from the conventional concepts and designs of the prior art, and in so doing provides an apparatus primarily developed for the purpose of permitting easy retrieval of the items.

### SUMMARY OF THE INVENTION

In view of the foregoing disadvantages inherent in the known types of article securing devices now present in the prior art, the present invention provides a new system for securing items to outerwear wherein the same can be easily mounted on and removed from the outerwear for permitting easy retrieval of the items.

In general, the present invention takes advantage of the metallic nature of garment clips and the magnetic attractiveness of magnets to secure the items on outerwear garments.

To attain this, the present invention in one aspect generally comprises a system for securing at least one item fashioned with a garment clip to an outerwear garment. The system comprises a first magnet for positioning exterior of the outerwear garment adjacent to the exterior face of the wall defining the outerwear garment and a second magnet for positioning in the interior of the outerwear garment adjacent to the interior face of the wall defining the outerwear garment. The second magnet is magnetically attracted to the first magnet. The garment clip affixed to the item desiring to be retained is also magnetically attracted to the first magnet, and is then affixed also to the first magnet with the force of both the first and second magnets combined.

In one preferred embodiment of the system, the garment clips are lighter and smaller for children's items, as children's items are generally smaller in size and lighter in weight.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

One significant advantage of the present invention is the ease with which the system is attached to outerwear garments and items without damaging the garments or items, or requiring alterations to the garments or items. The system is also easily removed from the garment and items, but remains securely attached to the garment and items unless the user applies significant force to the magnets to release the magnets from the garment clip, and thus the chance of unintended release from the outerwear garment is greatly reduced.

Further advantages of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

The invention will be better understood and objects of the invention will become apparent when consideration is given to the following detailed description thereof. Such description makes reference to the annexed drawings wherein:

FIG. 1 is a schematic perspective view of outerwear clothing that embodies the new system for securing items according to the present invention positioned thereon.

FIG. 2 is a schematic perspective view of elements of the present invention.

FIG. 3 is a schematic side sectional view of the present invention mounted on the wall of outerwear fabric.

### DESCRIPTION OF PREFERRED EMBODIMENTS

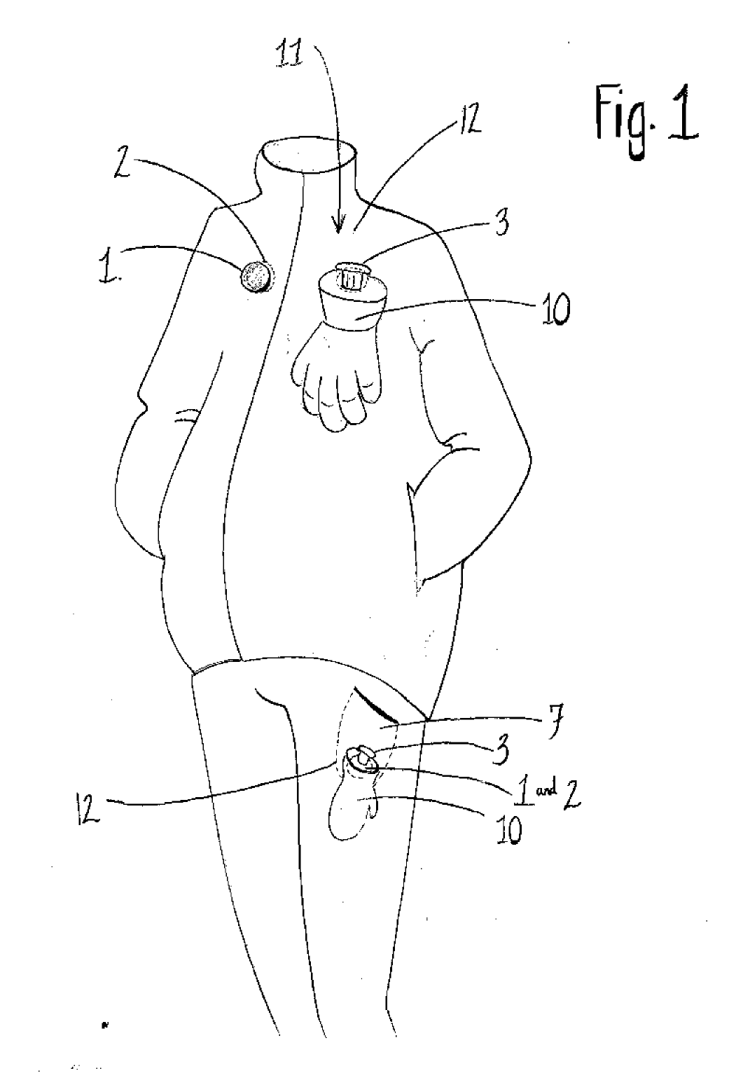

With reference now to the drawings, and in particular to FIGS. 1 through 3 thereof, a new system for securing items to outerwear garments embodying the principles and concepts of the present invention and generally designated by the reference numeral 11 will be described.

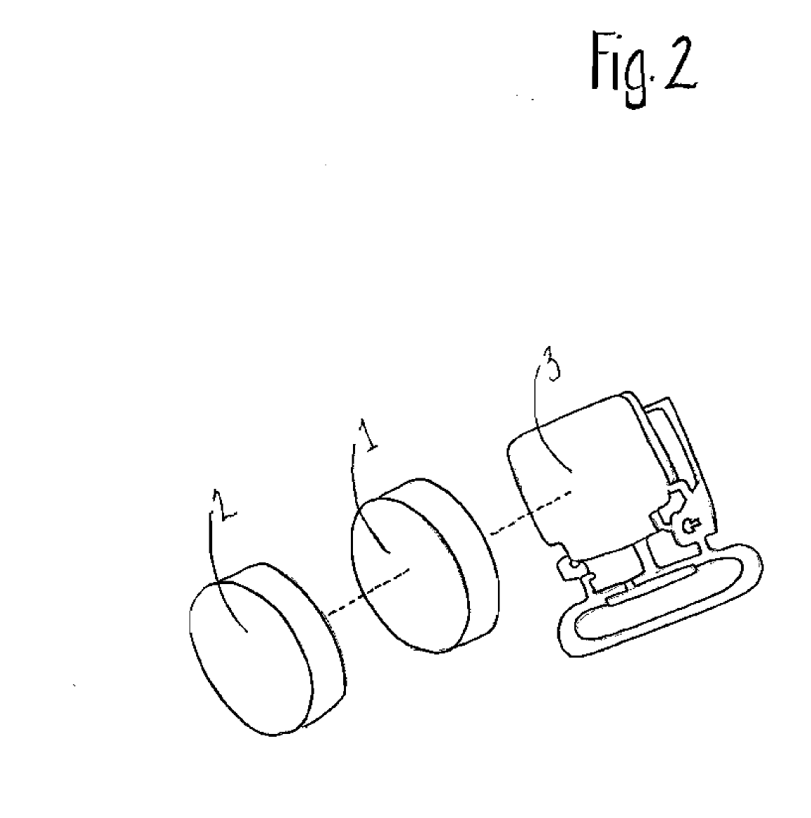

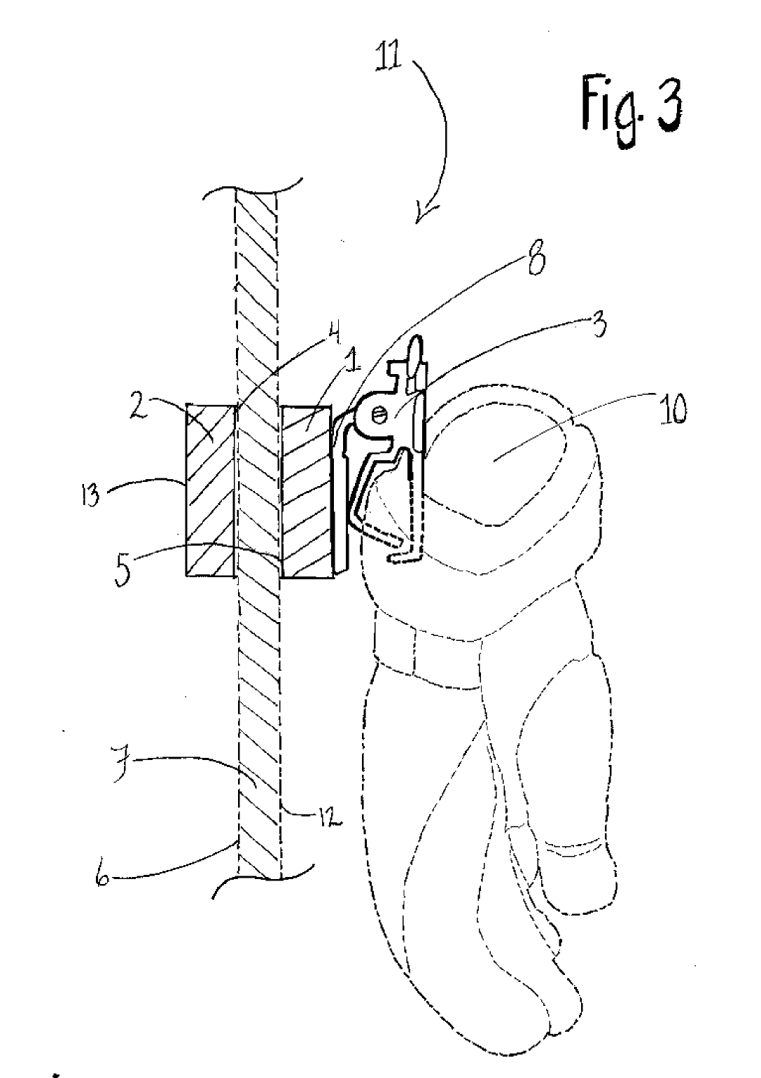

One aspect of the invention is directed to a system 11 for securing at least one garment clip 3 attached to a non-metallic item to be retained 10, such as a glove, mitten, goggles, etc . . . The exterior of the outerwear garment 12 may be at least partially defined by a wall 7 that has an interior face 6 corresponding to an interior 6 of the outerwear garment 7 and an exterior face 12 corresponding to an exterior 12 of the outerwear garment (see FIG. 3). The system 11 may generally include a first magnet 1 for positioning exterior of the outerwear garment 12 adjacent to the exterior face of the wall 12, a second magnet 2 for positioning in the interior garment wall 6 adjacent to the interior face 6 of the wall 7 (see FIGS. 2 and 3).

The second magnet 2 is magnetically attracted to the first magnet 1. The second magnet 2 may have a first side 9 which is substantially planar for abutting against the interior face 6 of the wall 7 of the outerwear garment. The second magnet 2 may also be substantially disc-shaped to also enhance the size of the first side 9 of the second magnet 2. The magnetic material employed for the first and second magnets should have suitably strong magnetic fields, and one suitable material is Neodymium-Iron-Boron (NdFeB), although other suitably strong materials could also be used. For example, the magnetic material could be Alnico and Ceramic/Ferrite types, although these types are typically less powerful and the effectiveness of the invention might be reduced. While it is possible that one of the first and second magnets could simply comprise a more weakly magnetized material such as iron or steel, such materials generally result in magnetic attraction that is not suitably and reliably strong enough to assure that the metallic item will remain secured to the second magnet until pulled from the second magnet by the user. In one embodiment of the invention, each of the first 1 and second 2 magnets has a diameter of approximately 20 mm (approximately 0.8 inches), which provides sufficient magnetic strength while not being overly bulky.

Optionally, the magnet or magnets may be coated. Optionally, the garment clip or clips may be of different shapes and sizes.

In implementation on the outerwear garment, the first magnet 1 is positioned exterior 12, or outside, of the outerwear garment to the exterior face 12 of the garment wall 7 that defines at least a portion of the outerwear garment, with the first side 9 of the first magnet 1 being abutted against the exterior face 12 of the wall of the garment. The second magnet 2 is positioned in the interior 6 of the garment 7 adjacent to the interior face 6 of the wall 7, with the second magnet 2 being magnetically attracted to the first magnet 1 through the wall 7. The first side 9 of the second magnet 2 is abutted against the interior face 6 of the wall 7. A garment clip 3 is attached to one non-metallic item, such as a glove or knitted hat 10, and is positioned on the exterior of the garment 12 and is removably attached to the second magnet 2 by magnetic attraction of the garment clip 3 to the second magnet 2.

The items fashioned with the garment clip may be brought into contact with the second magnet positioned in the interior of the purse such that the same is magnetically attracted to the second magnet. The magnetic attraction between the metal of the garment clip and the second magnet functions to hold the item(s) securely in place on the garment, while allowing the item(s) to be readily removed from the second magnet by the application of a firm pull on the item(s). The item(s) may be removed from the second magnet without removing the second magnet from the first magnet, so the first and second magnets remain in place on the wall of the garment.

With respect to the above description then, it is to be realized that the optimum dimensional relationships for the parts of the invention, to include variations in size, materials, shape, form, function and manner of operation, assembly and use, are deemed readily apparent and obvious to one skilled in the art in light of the foregoing disclosure, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### LIST OF REFERENCE NUMERALS FOR THE EMBODIMENTS OF THE DRAWINGS

1. First magnet
2. Second magnet
3. Garment clip
4. First side of second magnet
5. First side of first magnet
6. Interior of garment wall
7. Garment wall
8. Surface of garment clip
9. First side, or interior side, of second magnet
10. Item to be retained
11. System
12. Exterior of garment

I claim:

1. In combination: a piece of outerwear (coat, jacket, vest or pocket of a pants or skirt) having an interior and an exterior, the outerwear being at least partially defined by a wall having an interior face corresponding to an interior of the outerwear and an exterior face corresponding to an exterior of the outerwear; an item securing apparatus for securing items to the outerwear, comprising: a first magnet positioned exterior of the outerwear adjacent to the exterior face of the wall defining the outerwear; and a second magnet positioned in the interior of the outerwear adjacent to the interior face of the wall defining the outerwear, the second magnet being magnetically attracted to the first magnet through the wall of the outerwear; and

[symbol]

2. In combination: an item desiring to be retained (a glove, mitten, soft hat, goggles with a strap, etc.) will be fashioned with a metallic garment clip (also known as a mitten clip, pacifier clip or suspender clip) on its exterior, anywhere along the edge of the item or on a place where it fits snugly and securely,

3. The garment clip on the item desiring to be retained is brought close to the magnet on the exterior of the outerwear such that it affixes the item to the outerwear and allows the person wearing the outerwear hands-free movement and the security of safely carrying items without the risk of being tangled up in leashes or the pull of gravity making said items accidentally fall out of pockets.

4. Garment clips for children's items are of a lighter weight and size than the garment clips for affixing adult items to the exterior magnet, however, the two magnets that the items affix to are of equal size and strength both for adults and children.

5. The combination of claim 1 wherein the first side of the first magnet is substantially planar for abutting against the exterior face of the wall of the outerwear.

6. The combination of claim 1 wherein the second magnet has a first side which is substantially planar for abutting against the interior face of the wall of the outerwear.

7. The combination of claim 2 wherein the at least one item comprises a garment clip.

8. The combination of claims 3 and 4 wherein the garment clip has a front surface in the shape of a contoured design, and the contoured design resembles one object selected from a group of objects consisting of a circle, a hand shape, a star, a flower, a soccer ball, a basketball, a baseball, a rocket, a heart, a teddy bear, or other object, and may or may not be enameled with a graphic design upon it, and may or may not be left as plain metal in a regular shape of a garment clip.

9. A method of securing at least one item fashioned with a garment clip to a piece of outerwear that is at least partially defined by a wall having an interior face corresponding to an interior of the outerwear and an exterior face corresponding to an exterior of the outerwear where two magnets attract in opposite and adjacent locations to come together and subsequently attract the metal of the garment clip; the method comprising: providing an apparatus for securing at least one item to the outerwear, the apparatus including: a first magnet; a second magnet being magnetically attractable to the first magnet through the wall of the outerwear; a garment clip clipped to an item desiring to be retained such that when affixed to the first magnet, the first magnet on the exterior of the outerwear will be hidden. Once garment clip is affixed to the exterior magnet and the subsequent power of both magnets combined, it will appear such that the item desiring to be retained is ‘stuck’ to the chest or thigh area of the wearer of the outerwear.
